(19) United States
(12) Patent Application Publication
(10) Pub. No.: US 2003/0203424 A1
(43) Pub. Date: Oct. 30, 2003

(54) Method of detecting biological weapon use by monitoring a monolayer

(76) Inventor: Robert Neville O'Brien, Victoria (CA)

Correspondence Address: Robert N. O'Brien, 2614 Queenswood Dr., Victoria, BC, V8N 1X5, CA

(21) Appl. No.: 10/134,635

(22) Filed: Apr. 30, 2002

(51) Int. Cl. C12Q 1/04

(52) U.S. Cl. 435/34

(57) ABSTRACT

For a method of detecting biological weapon use, the disclosure suggests to apply known technology relating to interfacial films of monolayer-forming substances having molecules that when spread at an air/water interface will immerse into the water a hydrophilic terminal group for each molecule, with a long-chain carbon-containing hydrophobic group extending vertically upward away from the water. Such monolayers manifest monitorable properties which are susceptible using known instrumentation means to measurement of changes induced by biological contaminants thus making it possible to determine by appropriate testing what a bioattack signature change would be. Given siting of a monolayer coated aqueous body in a reservoir where a biological attack may occur, monitoring the monolayer properties for the signature change can detect whether and when such an attack does occur. One example of enacting the method involves a lake-sized water reservoir coated by a mixed monolayer which changes respecting color absorbance when bacteria and a biocide in the monolayer interact. Another example uses an instrumented film balance, ie., Langmuir trough as the reservoir, which is small enough for siting anywhere needed, eg., inside postal stations or other important buildings.

### BACKGROUND OF THE INVENTION

1. Technical Field

This invention relates to a method of detecting clandestine release at an attacked site of pathogenic bacteria used as a biological weapon. As described hereinafter, the method of detecting such an attack will involve a process of monitoring properties of an interfacial film known in surface chemistry as a ‘monomolecular film’ or ‘monolayer’, which is proposed to be formed on the surface of an aqueous body located anywhere deemed likely to be targeted for intentional contamination by release of pathogenic bacteria. What the monitoring specifically looks for shall be termed, in lexicography of the inventor, a ‘bioattack signature change’, meaning a technologically insensible change in hereinafter discussed monitorable monolayer properties that can be inferred with likelihood of correctness to occur as a result of intrusion of a non-naturally occuring quantity of pathogenic bacteria to the site of the aqueous body coated by the monolayer.

2. Background Information

Need for a method of detecting clandestine releases of pathogenic bacteria is especially apparent from the several anthrax fatalities that occurred in the United States late in 2001, establishing the dreadful fact that unauthorized individuals can somehow acquire sufficient quantities of anthrax (Baccillus anthracis) to produce lethal biological weapons.

Adoption of a universal anthrax immunization program like that instituted for military personnel during the Gulf War but terminated in 1998 seems unlikely thus far, and defense of civilian facilities against biological attacks is difficult because actual acts of releasing pathogenic bacteria will more often than not escape being witnessed directly. Obtaining verification that such an attack has occurred has tended to be postponed until after undue passage of time that increases lethality of infection. It is essential to administer antibiotics and anti-toxins to victims as soon as possible after the bacterial exposure event infecting them.

When no biological attack warning system is in place, inhalational anthrax victims tend to interpret first-stage disease symptoms, eg., fevers and pains, as an onset of flu; then, instead of seeking proper medical care, they may attempt self-medication using over-the-counter flu medications that are ineffective against the disease contracted. Even in the case where a presenting patient does seek professional medical care at the first sign of a flu-like symptom, likelihood of proper diagnosis of inhalation anthrax remains low unless some anthrax exposure preceding the symptoms is clinically suspected on some evidentiary indication, such as evidence that the workplace of the patient may have been contaminated.

Two-and-a-half years before the recent anthrax fatalities, the Working Group on Civilian Biodefense reported in a consensus statement in the Journal of the American Medical Association, May 12, 1999, that: “Currently, there are no effective atmospheric warning systems to detect an aerosol cloud of anthrax spores.” Technological non-existence of such a warning system after more than eight decades of anthrax weaponry research sponsored by several nations including Great Britain, the Soviet Union, the United States of America, and lately Iraq, constitutes unusual omission to devise something that all concerned with biological warfare have long had abundant reason to want devised. At least there is now a very recent report that a few pathogen sensor systems are finally under private development. In the March 2002 issue of Scientific American, in a short article by R. Casagrande entitled “Detecting Anthrax”, three different sensors known to be under development athough “not yet ready for deployment” are identified. The present inventor has insufficient information about the sensors mentioned by R. Casagrande to permit individual comment on them. Generally speaking, it would not be unlikely that a number of different operational modes based on different concepts may well prove independently workable, thus providing a selection of pathogen-sensing devices as useful alternatives to one another. Once proven suitable for actual deployment, the various makes of sensors are expected to compete in the marketplace as usual, where major purchase-decision factors include initial pricing, operational costs including re-supply of consumable materials, ease of instrument calibration, and ease of training personnel to use and service the new sensors effectively.

The three sensors of the abovecited Scientific American report are said to “incorporate cutting-edge technology”, a phrase which, in the present inventor's view, plausibly entails relatively higher cost purchase-decision factors by comparison to a case where a method of pathogen sensing may be found that does not so much depend on “cutting-edge technology” because it extensively uses well established techniques of scientific investigation, readily acquired materials, and off-the-shelf equipment to reduce pathogen detection costs.

The basic department of established technology to which the present inventor has turned to find a cost-effective biological attack warning method is the branch within surface chemistry which deals with ultra-thin films of surface-active chemicals at air-water interfaces, which are well known as ‘monomolecular films’ or ‘monolayers’. Surface-active organic compounds typically used for forming monolayers feature molecular structure with two unlike functional groups per molecule: a substantially water-insoluble, non-polar, long-chain hydrocarbon portion; and a short, terminal, hydrophilic polar portion. Placed at an air-water interface, a quantity of one of such dual-natured organic compounds—variously called ‘amphoteric’, ‘amphipathic’, or ‘amphiphillic’ compounds (depending on literature sources)—will form a stable molecularly oriented structure having hydrophilic portions of its molecules immersed into the aqueous subphase, eg., water, whereas non-polar water insoluble portions will align with one another and due to a hydrophobic effect will be directed away from the water, protruding into an overlying gas phase, eg., air. Molecules in the ideal case for such films as contemplated here will individually orient vertically, polar ‘head groups’ in the aqueous subphase, with the non-polar ‘tail groups’ rising vertically therefrom.

Hydrology and biochemistry have both seen extensive development of materials, techniques, and instruments pertaining to: first, coating aqueous bodies with monolayer-forming chemical films; and then, second, monitoring changes in properties of the monolayers which attend a wide variety of known causes. As will become apparent below, basic principles of several known monolayer-monitoring methods are readily adaptable to practicing the present invention.

Important background information to next briefly review concerns readily monitored monolayer properties or associated phenomena, with brief indication also of typical known means or instrumentation for such basic monolayer monitoring. The first monitorable phenomenon warranting mention is an optical phenomenon in sensible character but rooted in a fundamental viscous damping property known to be associated with monolayers, which also manifests itself in other sensible ways besides this phenomenon., here termed ‘image reflectance’ to distinguish it from other optical phenomena such as involve light absorbance and are studied using instruments of spectrometry, ellipsometry, and interferometry.

Image reflectance of a monolayer-coated liquid surface is, under the right conditions, manifested by strikingly different visual appearance from that of a surface of the same subphase liquid under the same conditions but not monolayer coated. In Retardation of Evaporation by Monolayers, V. K. LaMer, ed., (Academic Press, NY, 1962), photographs of Umberumberka Reservoir in various extents of being covered by a cetyl alcohol monolayer are labelled FIGS. 1-3 in the chapter by R. G. Vines entitled “Evaporation Control: A Method of Treating Large Water Storages”. The three photographs demonstrate that if weather cooperates, meaning no rain and a ripple-generating light wind not greater than about 8 kmh, areal extent of presence or absence of a monolayer on a sizable body of water can be gaged by simple visual inspection because a mirrorlike optical reflectance of the monolayer coated area will be evident, whereas small waves and ripples caused by light wind prevent similar mirrorlike reflectance of images by an uncoated area. The explanation for this is that the monolayer “exerts a strong damping effect on waves and ripples”, as R. G. Vines correctly states. For a laboratory setting, J. A. Mann, Jr. and R. S. Hansen devised instrumentation for artificial propagation of ripples and measurement of their velocity and attenuation as affected by monolayers. See Journal of Colloid Science 18, 757-771 and 805-819 (1963).

A point taught by the photos of Umberumberka Reservoir is that if the conditions permitting simple visual distinction between monolayer coated and uncoated areas are presumed constant, then it follows as expected that an introduced cause of monolayer deterioration would be inferrable from equally simple visual evidence, since an area losing its monolayer would revert to the uncoated appearance.

To clarify the same point, suppose that a monolayer-deteriorating agent were—unknown to a reservoir keeper—added to the aqueous subphase (underlying water) below the mirrorlike monolayer coated areas shown in the photographs of 250 acre Umberumberka Reservoir. Then, as the cetyl alcohol monolayer deteriorates, it would be expected that, given constancy of light ripple-generating wind, expansion of the uncoated ripple-covered area would proceed until there remains no more mirrorlike reflectance of any area. If it were assumed that all other factors possibly affecting the monolayer were innocuous in such a case, visual disappearance of the mirrorlike reflectance would give a reservoir keeper cause for alarm, justifying an inference or at least strong suspicion of likely contamination of the reservoir water by some monolayer-deteriorating agent. With long-chain alcohol monolayers, among the more likely to be considered candidate contaminants are bacterial organisms, since it is known that population explosions of certain of them can cause biodegradation of monolayer forming higher aliphatic alcohol materials which provide via their carbon content nutrition for the organisms.

The most typically and extensively monitored properties of small scale monolayers are the closely related interfacial tension and interfacial pressure, also called ‘surface tension’ and ‘surface pressure’ (or ‘film pressure’) in some literature. Accurate calculation of either interfacial tension or else interfacial pressure associated with a given monolayer can be accomplished by those of skill in the art on the basis of having accurately measured just the other property. Given a known area of interfacial film coverage, these properties are known to change with great sensitivity in response to changes in environmental conditions, including both weather parameters such as wind, air temperature, and relative humidity, and the intrusion of contaminants. Interfacial pressure is a force in the plane of a monomolecular film which is variable depending on extent of confinement of the film to a definite area covered. A laterally compressed or ‘condensed’ film exists when barriers bounding a monolayer coated area limit that area to an area smaller than would be spread across by the amount of film forming material used, in absence of the barriers. It is known that suppression of evaporation of water from the coated subphase is most effective for cetyl and stearyl alcohol films when they are in the condensed state.

Formation of an interfacial monolayer film characteristically causes a measurable reduction from the pre-existent interfacial tension an aquatic surface has had before its becoming monolayer coated, and surface chemistry takes advantage of this in highly developed research instruments with the capability of following changes in interfacial film properties. The most important class of instrument for monolayer investigations is generally called the ‘film balance’ the same thing formerly more commonly called a ‘Langmuir trough’. Practice of the method of the present invention is easily adapted to using a film balance as a small reservoir locatable where needed.

Numerous texts are available which teach what is known concerning use of film balances in monolayer investigations, and a recommended example is Chemistry at Interfaces by Finlay MacRitchie (Academic Press, 1990), which in parts of Chapters 4, 5, and 6 supplies much information respecting film balances and instrumentation thereof to investigate what shall here be termed the ‘monitorable monolayer properties’ of: interfacial pressure, interfacial tension, surface potential, interfacial rheology (viscoelasticity), surface temperature light absorbance and reflectance. The MacRitchie text includes many schematic figures of film balances set up with special instrumentation.

In Lipid and Biopolymer Monolayers at Liquid Interfaces, (Plenum Press, NY. 1989), K. S. Birdi credits the present inventor, R. N. O'Brien, and associates, with supplementing conventional methods of investigating monolayers by the use of laser interferometry techniques. “The effect of monolayers on the rate of evaporation of H₂O and solution of O₂ in H₂ O”, R. N. O'Brien et al, Canadian Journal of Chemistry, vol. 54, pp. 2739-44 (1976), describes how interferograms correlating with refractive index changes caused by surface temperature changes were made and used. More recently, issuance to the present inventor of U.S. Pat. No. 6,303,133 B1 for FILM-SPREADING POWDER FOR SUPPRESSING WATER EVAPORATION (Oct. 16, 2001) shows his ongoing involvement with monolayer technology. Cited in that patent was the thesis of A. I. Feher, “An Investigation of Monolayer Spreading Speeds at the Air/Water Interface”, (University of Victorian 1975), wherein certain quaternary ammonium halides were shown to have generally similar spreading pressures as higher aliphatic alcohols—a fact suggestive that a mixed monolayer using both these types of compounds is feasible.

As co-inventor with R. E. Jansson and S. Visaisouk of U.S. Pat. No. 5,104,649 (May 14, 1992) for SURFACE-FUNCTIONALIZED BIOCIDAL POLYMERS, the present inventor investigated the occlusion of rigid plastic surfaces with dead bacteria, when the plastic surfaces have biocidal agents bonded thereto. Such agents include amine salts quaternized in situ per the teachings of the Jansson et al patent. Distinguishing from such a case, the behaviour of quaternary ammonium halide biocides, if present in a monolayer at the surface of a bacterially contaminated aqueous body, would not involve similar surface occlusion since bonding of the biocide to a rigid plastic surface would not be present. Instead, the inventor expects that in a case of having the biocidal agent in a monolayer, killed bacteria would adhede together with bacteriocidal molecules, the latter tending to fall out of the monolayer into the aqueous subphase, thus altering the interfacial film.

Even setting aside the question whether this or that particular monolayer should or should not be equipped chemically for killing particular species of bacteria likely to contaminate the environment and enter the aqueous body, the following proposition would not be doubted by those of skill in monolayer technology: associated with any major intrusion of bacteria to the environment of an established monolayer, there would much more likely than not be a measurable change in one or more of its monitorable monolayer properties, that would attend the intrusion.

Insofar as the inventor is aware, applying the known technological tools for monolayer studies to provision of a method of detecting biological weapon use by monitoring a monolayer has never been previously suggested.

### BRIEF SUMMARY OF THE INVENTION

A major object of the invention is to achieve cost effectiveness for a method of detecting biological weapon use, by utilizing already well established monolayer-monitoring techniques, readily acquired substances that form monolayers, and off-the-shelf instrumentation, to the extent feasible. Although the invention per se has heretofore been unknown, the techniques, substances, and instruments that will suffice for its practice have in most cases been known for decades, both in the hydrology sector by those focussing their work on water conservation using monolayers to suppress evaporation, and in biochemistry by those focussing their work on measuring changes in monitorable monolayer properties attending reactions at interfacial films.

The present inventor's unique suggestion is to practice detection of catastrophic intrusion of pathogenic bacteria at a biologically attacked site, by monitoring a monolayer at the site in accordance with a method that features the five essential elements next set forth.

The first element is pre-placement at a site likely to be targeted for biological attack, if not naturally located there already, of a reservoir so holding an aqueous body exposed to the local atmosphere that it would be expected that a biological attack releasing substantial amounts of pathogenic bacteria at the site would result in contamination of the aqueous body held by the reservoir. In other words, the aqueous body is proposed for use as a means for collecting a sample of the bacterial agent used in the attack. The reservoir may be comparatively large, like Umberumberka Reservoir, or else small, as are laboratory film balances commonly used in monolayer research.

The second element is to establish a monolayer comprised of one or more suitable substances to form an interfacial film coating a significant portion of the surface of the aqueous body held in the pre-placed reservoir. Where there may be an intention to simultaneously conserve water in a large reservoir by suppressing evaporation therefrom, it would be desired to coat substantially the whole surface of the reservoir. However, for using a film balance solely for bioattack detection, advantages are described below for incompletely coating the aqueous body in the reservoir with the monolayer.

The third element is to utilize conventional means to monitor and, if feasible, control general environmental conditions affecting the reservoir site, including relative humidity, air and water temperature, velocity of air or water currents if present, spectral radiation intensity, and extent if any of possibly normal or endemic existence in the aqueous body of biological organisms. These are all conditions known capable of affecting measurements of monitorable monolayer properties, and may be regarded as factors capable of acting either individually or in concert to produce deterioration or loss of monolayer coatings. When practicing the method of the invention outdoors on a large scale, obviously, the aforesaid environmental conditions are not as readily controlled as they are monitored, whereas established technical means for their control as well as for being monitored may be resorted to when the method is practiced indoors at the small scale using film balances.

The fourth element is to provide suitable instrumentation of the established monolayer coated aqueous body in its reservoir to enact at least any one, but preferably more than one, of the known methods for measuring monitorable monolayer properties that have been mentioned in the BACKGROUND above and/or, equivalently, are known susceptible to measurement according to any prior art suggestions of instruments and research techniques.

The fifth element, which in concert with the third element confers collective operability for the purpose of the invention on the first, second, and fourth elements, is pre-use calibration of whatever monolayer monitoring instrumentation has been selected. Before commencing any actual, on-alert bioattack detection use, there necessarily will be comparatively less or more technical work involved to effect instrumentation calibration, depending on amount and nature of information already possessed regarding particular expectations about the consequences of different possible combinations of monolayer forming substances, aqueous body compositions, and bacteria species toward which a given implementation of the detection method of the invention may be directed. Scenarios for calibration include at one extreme a minimal extent of required effort when existing information entails that a given monolayer would be so degraded by bacterial contamination that an instrument reading indicating destruction of the monolayer would be expected within a few hours after contamination of the aqueous body by the bacteria.

At the other extreme regarding quantity of effort for calibration, there are instances where new system testing and instrumentation calibration using actual lethal bacterial samples of specific bacterial species will be needed. For example, in proposing a miniature reservoir that is essentially a film balance to be used in the vicinity of a building, the air circulation system of which is deemed a potential target for release, specifically, of anthrax spores used as a biological weapon, it should by now be well within routine capabilities for competent surface chemists., in collaboration with Center for Disease Control bacteriologists, to find the bioattack signature change attending anthrax interaction with a given monolayer. Duty of candor obliges the present inventor to admit not currently having factual data on such an interaction, primarily for reason he has not acquired a sample of Bacillus anthracis to use in calibrating film balance instrumentation adapted for biological attack detection employment. In the inventor's estimation, owing to the high calibre of surface chemists and bacteriologists, and high degree of monolayer technology development to date, acquisition of data to identify an anthrax—or for that matter any other—bacterial bioattack signature change does not invoke a requirement of undue experimentation beyond what can be done as a matter of routine by the right collaborators, ie., the surface chemists and bacteriologists who are reasonably expected to be those best suited to conducting the needed ‘signature-finding’ test implicit to the calibration of instrumentation proposed here as the fifth essential element of the method of the invention.

As apparent, size of an aqueous body to be coated by a monolayer may be large or small. Taking advantage of this fact, the invention makes feasible the mass-production of a large number of bioattack monitors that basically are specially adapted and instrumented film balances suitable for being located at urban sites associated with buildings deemed likely to be prospective targets of clandestine bioattack by terrorists. Locations just adjacent to air inlets to a building, and adjacent air duct openings inside a building constitute prime opportunities for rational monitor siting. At the same time, nothing inherent to the invention prevents its practice, if the environmental conditions permit, at the large outdoors size-scale, to monitor for possible catastrophic contamination of a public water storage reservoir.

In the wake of tragic terrorist attacks on America late in 2001, it is reasonable to contemplate possible attempts to contaminate large public water reservoirs, by a terrorist-piloted aircraft equipped for crop-dusting, for example. Detailed description of the invention to follow will commerce with illustrating one way to set up a large reservoir to monitor for such a bioattack. Further on, the detailed description will address the relatively miniaturized application of the method of the invention utilizing specially adapted film balances.

### BRIEF DESCRIPTION OF FIGURES OF DRAWING

FIG. 1 schematically illustrates, by an oblique perspective view, an outdoor lake-sized reservoir coated by a monolayer, with presence at the site of equipment set up in a system arranged to detect a color change that would result from a catastrophic bacterial intrusion.

FIG. 2 schematically illustrates, by an oblique perspective view, a table-top sized adaptation of a film balance used to practice the method of the invention indoors.

### DETAILED DESCRIPTION OF THE INVENTION

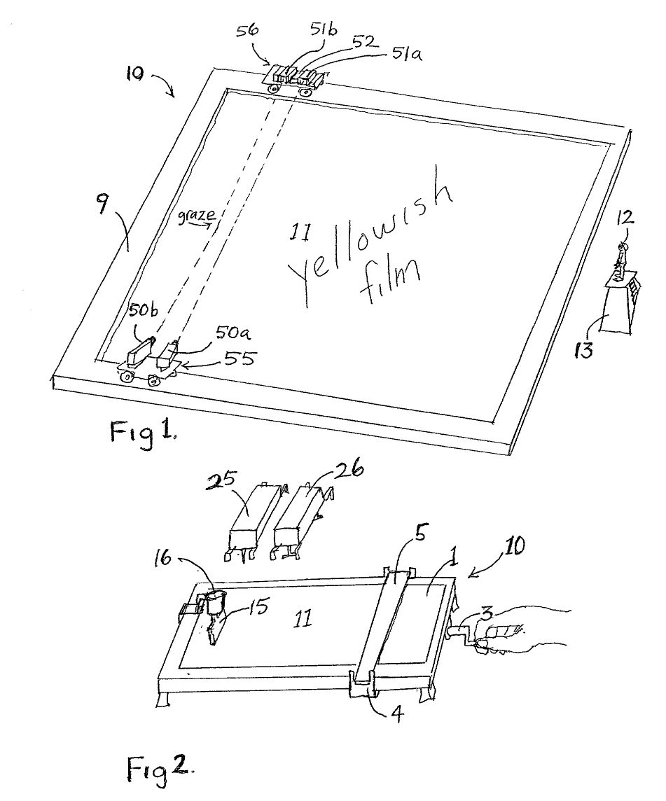

How the instant invention may be practiced at the larger end of a wide size range scale respecting an aqueous body-holding reservoir used will be explained with reference to FIG. 1. The lake-sized water reservoir shown is generally designated 10, is bounded by a flat-topped wall 9, and contains its aqueous body covered by a mixed monolayer 11 so composed that in sunlight the surface of the aqueous body would visually appear yellowish to the naked eye of observer 12 on a raised platform 13 to overlook reservoir 10. The yellowish appearance in this non-limiting example is due to providing that the mixed monolayer 11 be partly composed of a constituent containing iodine and which absorbs a substantial amount of the violet color spectral region of sunlight, namely, octadecyl trimethyl ammonium iodide. This substance is a member of the quaternary ammonium halide family of organic compounds that are simultaneously film-forming surfactants and biocidal. Bacteria contacting a biocide of this type tend to adhere to the biocide molecules.

The other constituent of which mixed monolayer 11 is composed preferably can be hexadecanol or octadecanol, a blend of these two higher fatty alcohols as has often been used for evaporation suppression, or even the inventor's recently patented evaporation suppressant composition that includes a minor amount of slaked lime powder with fatty alcohol.

In the usual manner, and for molecules of both constituents of film 11, comparatively long (16-18 carbon atoms) carbon chain portions will orient extended away from the bulk of the water of the film-coated reservoir. In a manner believed analogous to how hydrocarbon tails of hexadecyl trimethyl ammonium bromide (HDTAB) are thought by biochemists to arrange into spaces between cholesterol molecules in bilayer lipid membrane studies, octadecyl trimethyl ammonium iodide chains will similarly fit tails amongst those of monolayer-forming fatty alcohol molecules, thereby creating an interfacial monomolecular film of mixed constituents that coats an aqueous body in the usual manner with the polar or ionic head portions of molecules immersed.

Presence in film 11 of its special iodide relates causally to its yellowish coloration, since analytical chemistry finds this iodide to have broad bands centered at 360 and 290 nm, respectively near the edge of and inside the ultraviolet range. The yellowish color in daylight conditions may readily be sensed remotely by optical devices possibly carried by aircraft or even a Landsat type satellite, or more cheaply by a naked-eye observer 12 on platform 13. Automatic on-site monitoring to obviate need for a reservoir keeper on watch constantly is contemplated, and illustrated schematically by a set of violet-tuned lasers shown in FIG. 1 as 50a and 50b, with matching laser light receptors 51a and 51b disposed across the reservoir. Tuned lasers to monitor for a color change are within known technology and do not require detailed description. Basically, the FIG. 1 set-up calls for tuning the two lasers for violet sensitivity, utilizing laser 50a to produce a reference beam and 50b to produce a surface-grazing beam. Electronic balance means 52 connecting receptors 51a and 51b includes appropriate bridging and adjustment circuits (not shown), allowing system calibration to give a characteristic reading when monolayer film 11 is in the normal state of maintaining both the fatty alcohol and the iodine-containing constituents together in film 11.

Still referring to FIG. 1, the yellowish coloration due to iodide content of monolayer 11 would not be preserved for long after catastrophic reservoir contamination following an attacking overflight of reservoir 10 by a crop-dusting equipped aircraft dispersing any of the pathogenic bacterial species mentioned in the abovecited Jansson et al patent and susceptible to biocidal effect of quaternary amines. The major bacterial intrusion from such an attack would encounter the biocide constituent of monolayer 11, which preferably may constitute from about ten to thirty percent of the total amount of monolayer forming substances utilized. Each bacterium contacting a biocide molecule would tend to adhere thereto and the pair, or clumps of such pairs, as a result of interaction between bacteria and biocide, would tend to exit monolayer 11 by falling therefrom into the underlying bulk aqueous phase. This removal mechanism is causative of loss of the yellowish surface coloration because of physical removal of the iodide content of the biocide away from the ultrathin interfacial region grazed by the beam from laser 50b. Absent the iodide constituent at the interface, there would of course be more violet received by receptor 51b, ie., less attenuation of violet transmitted by reflection after grazing, less violet being absorbed in the grazing process when the iodide is no longer present in monolayer 11. Given a set up for automatically continuous recording of the difference between the two beams at their receptors, any major departure from the normal difference when the monolayer 11 is constituted as originally spread is a signal of change thereof that would be cause for a reservoir keeper to suspect a contaminating attack. The loss of coloration, detectable optically whether by means of human visual observation or using instruments in known ways, would constitute the looked-for signature change indicating a significant likelihood that a bioattack attempting to contaminate the water of reservoir 10 has occurred.

Degree of effectiveness of this particular method of practicing the invention would vary depending on local weather factors, which must at least be such as permit maintaining monolayer 11 on reservoir 10. Not controlled, general environmental conditions capable of causing major alteration of the film 11 must at least be themselves monitored so that allowances can be made in order to avoid false automatic indications of a bioattack. Rain or strong wind breaking up monolayer 11 could also cause loss of yellowish coloration and thus indication by the tuned laser system of a catastrophic change.

For another measure to avoid false signals, water purity monitoring should be practiced to be sure that film deterioration is not being caused by population explosions of naturally occurring Pseudomonas or Flavobacterium species that may first be attracted to the higher fatty alcohol containing constituent, about 70-90% of a mixed monolayer 11, as a source of nutrition to them, but which would then be decimated by the biocide constituent.

Given favorable or at least innocuous environmental conditions, including all those specifically mentioned in the SUMMARY above where the third essential element of the method of the invention was specified, the method can be practiced along the lines basically described herein with reference to FIG. 1 and thereby contribute a significant measure of civilian defense preparedness in face of possible bioattacks on large public water reservoirs. Considering the advisability in general that someone be employed on a regular basis to look after basic security of a large reservoir holding public drinking water, the option of using a human reservoir keeper/observer 12 periodically mounting a platform 13 as shown in FIG. 1 is recommended.

A final detail of FIG. 1 to discuss is use of mobile platforms generally indicated as 55 and 56 to support the components of the laser-based optical monitoring system illustrated and already explained. Assuming a fairly large reservoir, it is conceivable that a biological attack attempting its contamination from the air might not have the causative consequence of a film loss of the violet-absorbing constituent evenly operative across the the whole area of water surface in the reservoir. Instead, a patch of loss in a particular smaller region contaminated by airdropped bacteria might occur, in which case, unless that affected region were grazed by the grazing beam of laser 50b the desired warning signal of contamination occurrence would not be forthcoming. This problem is easily resolved, however, by so moving the system components as to check for film loss across the whole reservoir surface, conceived as adjacent band regions to be successively grazed. Additionally, mobile platforms 55 and 56 obviously would facilitate moving the reservoir monitoring system of FIG. 1 to another reservoir if desired. It will be understood that above description in detail of embodying or practicing the method of the invention in accordance with what FIG. 1 illustrates does not limit the invention and is exemplary only.

Turning now to embodying or practicing the method of the invention at the smaller size-scale of relatively compact units of apparatus usable virtually wherever a biological attack might be expected to occur, FIG. 2 provides a second instructive illustration which again is not intended to limit the invention specifically to what is shown. The inventor makes no claim of uniqueness of physical features of the film balance here generally designated 10 and holding an aqueous body 1 coated in the indicated region by monolayer 11 but uncoated in the region indicated by the lead line for the figure element numeral 1. Movable barrier 5 separates the filmed (monolayer coated) and nonfilmed (uncoated) surface regions in the typical manner of most film balances, also called Langmuir troughs. Carriage means 4 to support and controllably move barrier 5 is actuated by wormdrive 3 in conjunction with which a revolution counter (not shown) would be helpful in calculating interfacial area coated by monolayer 11 at a given pressure.

For measurement of the fundamental interfacial pressure and tension properties of monolayer 11, a Wilhelmy plate 15 dipped into aqueous body 1 through monolayer 11 is provided. To measure downward pull on plate 15 by fluid at the penetrated interface, a force transducer 16 is shown with the plate 15 suspended therefrom. Plate 15 and transducer 16 are preferably combined in a portable unit that for use is rested atop the film balance 10 as shown but can be removed therefrom easily for transfer to an identical film balance located elsewhere.

FIG. 2 is partly exploded in the sense that the two rectangular portable, unitary, instrumentation means 25 and 35, like the plate 15 and transducer 16 unit, are shown removed from resting positions where they can be individually placed atop the film balance 10 at desired times. Instrument 25 is intended to represent a surface potential meter, and instrument 26 is intended to represent a viscosimeter. Alternatively, instrument 26 may also represent a unit combining the capillary ripple generating and ripple sensing probes described in the Mann, Jr. and Hansen articles cited above, wherein measurement of ripple amplitude attenuation associated with viscoelastic properties of a monolayer has been well taught and described.

Further, an advantageous hydromechanical feature that also is nothing new is assumed present when needed although not shown, namely, some suitable submerged means for stirring and distributing non-motile bacteria within the bulk of aqueous body 1, so that such bacteria which do not have the capability of locomotion can be forced into contact with the underside of monolayer 11 after having fallen onto the uncoated region of the surface of aqueous body 1. Such stirring means facilitate an established biochemical investigations technique which has been briefly and clearly described in a passage of the “Lipids in Water” chapter by Morris B. Abrahamson in The Chemistry of Biosurfaces, Michael L. Hair, ed., Marcel Dekker Inc. (New York, 1971) reading as follows: “The substance of biological interest (proteins, anaesthetics, or antibiotics) in an appropriate solvent is injected behind the movable float into the aqueous phase. By stirring, it is brought to the region below the film where it may react, and changes in surface pressure or potential measure the extent of the reaction.”

For the purpose of now systematically reviewing the five essential elements of the method of the present invention, with use supposed of a film balance type small size scale use-setting referenceable to FIG. 2, it is proposed that the substance of biological interest in this instance may be the pathogen, Bacillus anthracis, and that the monolayer here to be used is not of mixed composition but is instead 100% octadecyl trimethyl ammonium iodide.

A reservoir holding an aqueous body exposed to the local atmosphere is needed to be located at a site deemed likely to be targeted for biological attack. Since the film balance 10 of FIG. 2 is such a reservoir, let it be placed near an air duct outlet into a room inside a postal station, thereby satisfying the first essential element of the method of this invention. It is reasonable to expect that a biological attack releasing a substantial quantity of anthrax spores to travel with moving air through the ductwork until coming out at the air outlet would result in contamination of the aqueous body 1 held by film balance 10, a smallish reservoir.

The second essential element of the method is to establish a monolayer forming an interfacial film coating a significant portion of the surface of the aqueous body held in the pre-placed reservoir.

Here there is little if any importance attached to evaporation suppression since the small amount of liquid for aqueous body 1 can and likely should be replaced every so often on a regular schedule. The common use of higher fatty alcohols to form evaporation suppressant monolayers may here be disregarded and a 100% biocidal monolayer forming substance shall be used instead, namely, octadecyl trimethyl ammonium iodide, sufficing to satisfy the second essential element of the method of the invention, which it should be noted does not require total coating of the entire surface of the aqueous body, but only a significant portion thereof. Areal size of that portion is adequate if it conveniently allows placement and use of the needed instrumentation to be used to measure monitorable monolayer properties.

There is need here to monitor and control general environmental conditions affecting the sited film balance 10, including relative humidity, air and water temperature, velocity of air moving in the vicinity and of liquid when stirred, spectral radiation intensity, and extent if any of extraneous biological organisms, which are all clearly conditions respecting which established technical means for their control as well as for being monitored may be resorted to without need for developing new technology, when the method is practiced, as here, indoors and at a small scale, since film balance 10 of FIG. 2 provides the reservoir. Provision of environmental conditions controls of purely conventional character here satisfies the third essential element of the method of the invention.

The fourth element requires provision of suitable instrumentation for enacting at least one known method for measuring monitorable monolayer properties that were mentioned in the BACKGROUND above and/or, equivalently, are known susceptible to measurement according to any prior art suggestions of instruments and monolayer research techniques. Here it is apparent from FIG. 2 that three properties-measuring methods are preferable to use and easy to practice: using Wilhelmy plate 15 and associated transducer 16 to measure interfacial tension and pressure in the usual way; using similarly known instrumentation to measure surface potential; and also using means for measuring the viscoelastic (rheological) interface property, thereby amply satisfying the fourth element of the method of the invention.

It will be recalled that the fifth element, which in concert with the third element confers collective operability for the purpose of the invention on the first, second, and fourth elements, is pre-use calibration of whatever monolayer monitoring instrumentation has been selected. Given that the three examples of instrumentation represented in FIG. 2 are highly familiar to those who use film balances in surface chemistry research, their calibration requirements can be met by following conventional procedures. All then that remains to be done before actual on-alert bioattack detection use of apparatus shown in FIG. 2 is the acquisition beforehand of data for monitorable monolayer property changes in monolayer 11 in film balance 10 that would be characteristic for and amount to the bioattack signature change for the case of contamination of aqueous body 1 by Bacillus anthracis.

To acquire the pertinent pressure, tension, potential, and other data for the reaction of Bacillus anthracis bacteria with monolayer 11, eg. octadecyl trimethyl ammonium iodide, requires obtaining a sample of this dangerous species with which to contaminate aqueous body 1. Stirring in the usual way may be used to introduce the reactant beneath the monomolecular film 11 of film balance 10 of FIG. 2. Such testing to supply needed data does not invoke an undue or non-routine aspect of experimentation, which instead is essentially routine, notwithstanding lethality of the species.

The inventor considers it far more likely than not that a distinctive set of data for the parameters measured will show up, which subsequently can be monitored for, starting an on-watch usage of film balance 10 with a fresh uncontaminated aqueous body 1 and established monolayer 11. It should be noted that the question of extent of ‘kill rate’ by disinfection is not here the issue. The invention is not purported to be a means for complete destruction of the contaminating bacteria; rather, the aim is to provide a cost effective biological attack detection system. Each biocide varies with regard to ‘kill rates’ for different species, in different general conditions of humidity and temperature, and so forth. A bioattack signature change in a monolayer subjected to pathogenic contamination is expected to be found even if all bacteria do not die in the monitoring apparatus. Finally, the illustrative examples discussed by reference to FIGS. 1 and 2 are not meant to specifically limit the invention. Only the appended claims do.

As co-inventor with R. E. Jansson and S. Visaisouk of U.S. Pat. No. 5,104,649 (May 14, 1992) for SURFACE-FUNCTIONALIZED BIOCIDAL POLYMERS, the present inventor investigated the occlusion of rigid plastic surfaces with dead bacteria, when the plastic surfaces have biocidal agents bonded thereto. Such agents include amine salts quaternized in situ per the teachings of the Jansson et al patent. Distinguishing from such a case, the behaviour of quaternary ammonium halide biocides, if present in a monolayer at the surface of a bacterially contaminated aqueous body, would not involve similar surface occlusion since bonding of the biocide to a rigid plastic surface would not be present. Instead, the inventor expects that in a case of having the biocidal agent in a monolayer killed bacteria would adhede together with bacteriocidal molecules, the latter tending to fall out of the monolayer into the aqueous subphase, thus altering the interfacial film.

Even setting aside the question whether this or that particular monolayer should or should not be equipped chemically for killing particular species of bacteria likely to contaminate the environment and enter the aqueous body, the following proposition would not be doubted by those of skill in monolayer technology: associated with any major intrusion of bacteria to the environment of an established monolayer, there would much more likely than not be a measurable change in one or more of its mnonitorable monolayer properties, that would attend the intrusion.

Insofar as the inventor is aware, applying the known technological tools for monolayer studies to provision of a method of detecting biological weapon use by monitoring a monolayer has never been previously suggested.

What is claimed is:

1. A method for, detecting biological weapon uses comprising as essential elements:
first, locating, at a site deemed likely to be attacked by biological weapon use, a reservoir holding an aqueous body capable of being contaminated by bacterial intrusion from use of the biological weapon;
second, establishing as an interfacial film coating said aqueous body a monolayer formed by a suitable organic compound having a polar or ionic terminal group that immerses into the aqueous body, and a hydrophobic carbon containing chain that tends to project upwardly from the terminal group;
third, monitoring and if feasible controlling, by usual means, any general environmental conditions in the vicinity of said reservoir that are apt to effect alterations in said monolayer that could be confused with alterations due to contamination by bacterial intrusion from use of a biological weapon;
fourth, providing instrumentation means capable of measuring monitorable monolayer properties, in particular as said properties would tend to change as a consequence of contamination by bacterial intrusion from use of a biological weapon; and,
fifth, calibrating said instrumentation so that a difference is evident in measured data comparing the cases of no bacterial intrusion from use of a biological weapon and of occurrence of said bacterial intrusion, data for the latter being monitored for as a distinctive bioattack signature change that can be pre-identified by routine pre-use testing of a monolayer monitoring system subjected to the effect of a deliberately introduced pathogenic bacteria sample.

2. The method of claim 1 wherein said reservoir is a lake-sized public water supply reservoir.

3. The method of claim 2 wherein said monolayer is a mixed monolayer essentially composed of from about 70-90% higher aliphatic alcohol and from about 10-30% octadecyl trimethyl ammonium iodide.

4. The method of claim 1 wherein said reservoir is a film balance typical of small reservoirs used for monolayer investigations in laboratory research by surface chemists.

5. The method of claim 4 wherein said monolayer is composed of 100% octadecyl trimethyl ammonium iodide.

6. The method of claim 1 wherein said monitorable monolayer properties to be measured by said instrumentation means are selected, either individually in the alternatives or else preferably in combination to constitute a relatively more comprehensive set of properties, from among the properties of monolayers consisting of interfacial pressures, interfacial tension, surface potential, interfacial viscosity, viscoelasticity, optical absorbance, reflectance, and surface temperature;
whereby an expected extent of definiteness that a biological attack is what measured changes in monitorable monolayer properties actually indicate will tend to be greater, the greater is the number of properties monitored for the same monolayer, so that said bioattack signature change is rendered more accurate.
